SEQUENCE LISTING (45 sequences)

SEQ ID NO: 1 - protein, 188 aa, Nocardiopsis prasina
ADIIGGLAYTMGGRCSVGFAATNAAGQPGFVTAGHCGRVGTQVTIGNGRGVFEQSVFPGNDAAFVRGTSNFTLTNLVSRYNTGGYATVAGHNQAPIGSSVCRSGSTTGWHCGTIQARGQSVSYPEGTVTNMTRTTVCAEPGDSGGSYISGTQAQGVTSGGSGNCRTGGTTFYQEVTPMVNSWGVRLRT

SEQ ID NO: 2 - protein, 6 aa, Nocardiopsis prasina
SEDKPF

SEQ ID NO: 3 - protein, 7 aa, Nocardiopsis prasina
FVDAQPK

SEQ ID NO: 4 - protein, 8 aa, Nocardiopsis prasina
SAQAVEKL

SEQ ID NO: 5 - protein, 8 aa, Nocardiopsis prasina
ISSEDKPF

SEQ ID NO: 6 - protein, 8 aa, Nocardiopsis prasina
SRDPIYSN

SEQ ID NO: 7 - protein, 8 aa, Nocardiopsis prasina
NQRSPQLQ

SEQ ID NO: 8 - protein, 8 aa, Nocardiopsis prasina
AENNQRNF

SEQ ID NO: 9 - protein, 9 aa, Nocardiopsis prasina
SRDPIYSNK

SEQ ID NO: 10 - protein, 9 aa, Nocardiopsis prasina
VNNDDRDSY

SEQ ID NO: 11 - protein, 11 aa, Nocardiopsis prasina
EITPEKNPQLR

SEQ ID NO: 12 - protein, 12 aa, Nocardiopsis prasina
FEITPEKNPQLR

SEQ ID NO: 13 - protein, 13 aa, Nocardiopsis prasina
SREEGQQQGEQRL

SEQ ID NO: 14 - protein, 7 aa, Nocardiopsis prasina
FVDAQPQ

SEQ ID NO: 15 - protein, 9 aa, Nocardiopsis prasina
SAQDVERLL

SEQ ID NO: 16 - protein, 11 aa, Nocardiopsis prasina
PGSAQDVERLL

SEQ ID NO: 17 - protein, 13 aa, Nocardiopsis prasina
AFPGSAQDVERLL

SEQ ID NO: 18 - protein, 6 aa, Nocardiopsis prasina
AEFGSL

SEQ ID NO: 19 - protein, 7 aa, Nocardiopsis prasina
VSIIDTN

SEQ ID NO: 20 - protein, 7 aa, Nocardiopsis prasina
PEEVIQH

SEQ ID NO: 21 - protein, 8 aa, Nocardiopsis prasina
IQQGKGIF

SEQ ID NO: 22 - protein, 9 aa, Nocardiopsis prasina
PEEVIQHTF

SEQ ID NO: 23 - protein, 10 aa, Nocardiopsis prasina
DGELQEGRVL

SEQ ID NO: 24 - protein, 10 aa, Nocardiopsis prasina
NALKPDNRIE

SEQ ID NO: 25 - protein, 11 aa, Nocardiopsis prasina
ALPEEVIQHTF

SEQ ID NO: 26 - protein, 11 aa, Nocardiopsis prasina
SLENQLDQMPR

SEQ ID NO: 27 - protein, 12 aa, Nocardiopsis prasina
NALPEEVIQHTF

SEQ ID NO: 28 - protein, 5 aa, Nocardiopsis prasina
DTSNF

SEQ ID NO: 29 - protein, 6 aa, Nocardiopsis prasina
LDTSNF

SEQ ID NO: 30 - protein, 7 aa, Nocardiopsis prasina
ADFYNPK

SEQ ID NO: 31 - protein, 8 aa, Nocardiopsis prasina
HENIARPS

SEQ ID NO: 32 - protein, 8 aa, Nocardiopsis prasina
NSQHPELK

SEQ ID NO: 33 - protein, 9 aa, Nocardiopsis prasina
LHENIARPS

SEQ ID NO: 34 - protein, 9 aa, Nocardiopsis prasina
NNQLDQTPR

SEQ ID NO: 35 - protein, 10 aa, Nocardiopsis prasina
NTNEDIAEKL

SEQ ID NO: 36 - protein, 8 aa, Nocardiopsis prasina
NSQHPELQ

SEQ ID NO: 37 - protein, 10 aa, Nocardiopsis prasina
NTNEDTAEKL

SEQ ID NO: 38 - protein, 11 aa, Nocardiopsis prasina
RSPDDERKQIV

SEQ ID NO: 39 - protein, 6 aa, Nocardiopsis prasina
SIIDTN

SEQ ID NO: 40 - protein, 7 aa, Nocardiopsis prasina
SQSDNFE

SEQ ID NO: 41 - protein, 6 aa, Nocardiopsis prasina
DFYNPK

SEQ ID NO: 42 - protein, 8 aa, Nocardiopsis prasina
LDQTPRVF

SEQ ID NO: 43 - protein, 10 aa, Nocardiopsis prasina
NALEPDHRVE

SEQ ID NO: 44 - protein, 8 aa, Nocardiopsis prasina
LDQNPRVF

SEQ ID NO: 45 - protein, 11 aa, Nocardiopsis prasina
GNPDIEHPETM

(19) United States
(12) Patent Application Publication
(10) Pub. No.: US 2011/0097448 A1
(43) Pub. Date: Apr. 28, 2011

(54) Protein Hydrolysate Compositions Stable Under Acidic Conditions

(75) Inventors: Theodore M. Wong, Ballwin, MO (US); Phillip S. Kerr, Wildwood, MO (US); Parthasarathi S. Ghosh, Chesterfield, MO (US); Jason F. Lombardi, Edwardsville, IL (US); Gitte B. Lynglev, Frederiksberg (DK); Tine Hoff, Holte (DK); Lars LH Christensen, Alleroed (DK); Peter R. Oestergaard, Virum (DK)

(73) Assignee: SOLAE, LLC, St. Louis, MO (US)

(21) Appl. No.: 12/996,565

(22) PCT Filed: Jun. 19, 2009
(86) PCT No.: PCT/US09/48025

§ 371 Date: Dec. 6, 2010

Related U.S. Application Data
(60) Provisional application No. 61/074,543, filed on Jun. 20, 2008.

(51) Int. Cl. A23J 1/00; C12P 21/06; A23J 1/08; A23J 1/20; A23L 2/66; C12G 3/00; A23L 2/02; A23C 9/152; A23C 9/18; A23C 9/20; A23J 1/14; A23C 11/10; A23F 5/00; A23L 1/302; A23L 1/305; C11B 5/00; A23L 1/22

(52) U.S. Cl. 426/72; 435/68.1; 426/656; 426/657; 426/614; 426/580; 426/590; 426/592; 426/599; 426/588; 426/634; 426/594; 426/593; 426/654; 426/541; 426/650

(57) ABSTRACT

The present invention provides protein hydrolysate compositions, processes for making protein hydrolysate compositions, and food products comprising protein hydrolysate compositions. The protein hydrolysate compositions generally comprise a mixture of oligopeptides having an average molecular size of less than about 10,000 Daltons, wherein at least about 60% of the oligopeptides are soluble at a pH of less than about 7.0.

### FIELD OF THE INVENTION

The present invention relates to protein hydrolysate compositions that are stable at acid pH levels, processes for making protein hydrolysate compositions that are stable at acid pH levels, and food products comprising protein hydrolysate compositions that are stable at acid pH levels.

### BACKGROUND OF THE INVENTION

The rates of obesity and the diseases associated with obesity are rising in the Unites States and throughout the world. While there is no single underlying cause, a contributing factor may be the fast-paced, harried life styles of many individuals and the concomitant consumption of fast food. Most fast food tends to be high in fat and/or sugar. There is a need, therefore, for a nutritious, ready accessible food product that can be eaten or drunk “on the go.” This food product should not only taste good, but it should also be nutritionally sound; that is, the product should be low in fat, high in protein, and high in vitamins and antioxidants.

While soy is an excellent source of protein, it tends to have “grassy” or “beany” flavors that some individuals find objectionable or unpalatable. What is needed, therefore, is an isolated soy protein product with reduced “soy” flavors and reduced bitterness or astringency. Furthermore, if the desirable food product is a liquid beverage, then the isolated protein product to be added to the liquid beverage ideally should be clearer or more transparent that the starting material, i.e., the isolated protein product should have a high degree of solubility. Additionally, the isolated protein product should be stable at the pH of the desired liquid beverage.

### SUMMARY OF THE INVENTION

One aspect of the present invention provides a protein hydrolysate composition. The protein hydrolysate composition comprises a mixture of oligopeptides having an average size of less than about 10,000 Daltons. Furthermore, the protein hydrolysate composition has a degree of hydrolysis of at least about 2.5%, generally at least about 5.0%, preferably at least about 7.5%, and most preferably at least about 10% and a solid solubility index of at least about 60% at a pH value of less than about pH 7.0.

Another aspect of the invention encompasses a process for preparing a protein hydrolysate composition. The process comprises contacting a protein material with at least one endopeptidase that cleaves peptide bonds of the protein material to form a mixture of oligopeptides having an average size of less than about 10,000 Daltons, wherein the mixture of oligopeptides comprises the protein hydrolysate composition. The process further comprises lowering the pH of the protein hydrolysate composition to a value of less than about pH 7.0, wherein the protein hydrolysate composition has a solid solubility index of at least about 60% and a degree of hydrolysis of at least about 2.5%, generally at least about 5.0%, preferably at least about 7.5%, and most preferably at least about 10%.

A further aspect of the invention provides a food or beverage product comprising a protein hydrolysate composition. The protein hydrolysate composition comprises a mixture of oligopeptides having an average size of less than about 10,000 Daltons, and the composition has a degree of hydrolysis of at least about 2.5%, generally at least about 5.0%, preferably at least about 7.5%, and most preferably at least about 10% and a solid solubility index of at least about 60% at a pH of less than about 7.0.

Other aspects and features of the invention will be in part apparent and in part pointed out hereinafter.

### DESCRIPTION OF THE FIGURES

FIG. 1 is a plot of the percent of soluble solids as a function of the degree of hydrolysis (%) of hydrolysates generated with serine protease 1 (SP1, circles) or ALCALASE® (ALC, squares).

FIG. 2 depicts the percent of soluble solids of various hydrolysates as a function of pH. The figure presents the percent of soluble solids for a 5% ALC hydrolysate (diamonds), a 10% ALC hydrolysate (squares), and a 5% ALC hydrolysate (triangles) as a function of pH.

FIG. 3 presents the diagnostic scores for various sensory attributes of SP1 or ALCALASE® (ALC) hydrolysates. Panel A presents the difference from control (i.e., SP1 sample) of the indicated hydrolysates that were presented to the assessors as slurries in water with 2.5% solids at neutral pH. Panel B depicts the difference from control (i.e., HXP 212) of the indicated hydrolysates that were presented to the assessors as 2.5% slurries in water. Panel C presents the difference from control (i.e., HXP 212) of the indicated hydrolysates that were presented to the assessors in orange sport beverages at pH 3.0 at 1.6% protein. Panel D depicts the difference from control (i.e., HXP 212) of the indicated hydrolysates that were presented to the assessors in orange sport beverages at pH 3.8 at 1.6% protein.

FIG. 4 depicts the molecular weight distribution of peptide fragments in SP1 (light gray bars) and ALCALASE® (ALC) (dark gray bars) hydrolysates.

### DETAILED DESCRIPTION OF THE INVENTION

It has been discovered that cleaving a protein material with certain endopeptidases produces a protein hydrolysate composition comprising a mixture of oligopeptides, wherein the oligopeptides are soluble at acidic pH levels. These protein hydrolysate compositions also have improved flavor profiles and sensory attributes relative to those of the starting protein material. Protein hydrolysate compositions having these properties are stable at acid pH levels and may be useful as supplements to ready-to-drink beverages and other food products.

### I. Process for Preparing a Protein Hydrolysate Composition

One aspect of the present invention provides a process for preparing a protein hydrolysate composition comprising a mixture of oligopeptides having an average size of less than about 10,000 Daltons, wherein the composition has a degree of hydrolysis of at least 2.5%, generally at least about 5.0%, preferably at least about 7.5%, and most preferably at least about 10% and a solid solubility index of at least 60% at less than about pH 7.0. The process comprises contacting a protein material with at least one endopeptidase, wherein the protein material is hydrolyzed to form a mixture of oligopeptides. The process further comprises lowering the pH of the hydrolysate to less than about pH 7.0.

a. Hydrolytic Cleavage

The first step of the process comprises cleaving the protein material into a mixture of smaller sized oligopeptide fragments. In general, the protein material is contacted with at least one endopeptidase to form the mixture of oligopeptides. Examples of suitable protein materials and suitable endopeptidases are detailed below.

i. Protein Material

In some embodiments, the protein material may be a soy protein material. A variety of soy protein materials may be used in the process of the invention to generate a soy protein hydrolysate. In general, the soy protein material may be derived from whole soybeans in accordance with methods known in the art. The whole soybeans may be standard soybeans (i.e., non genetically modified soybeans), genetically modified soybeans (such as, e.g., soybeans with modified oils, soybeans with modified carbohydrates, soybeans with modified protein subunits, and so forth) and combinations thereof. Suitable examples of soy protein material include soy extract, soymilk, soymilk powder, soy curd, soy flour, soy protein isolate, soy protein concentrate, and mixtures thereof.

In an iteration of this embodiment, the soy protein material used in the process may be a soy protein isolate (also called isolated soy protein, or ISP). In general, soy protein isolates have a protein content of at least about 90% soy protein on a moisture-free basis. The soy protein isolate may comprise intact soy proteins or it may comprise partially hydrolyzed soy proteins. The soy protein isolate may have a high content of storage protein subunits such as 7S, 11S, 2S, etc. Non-limiting examples of soy protein isolates that may be used as starting material in the present invention are commercially available, for example, from Solae, LLC (St. Louis, Mo.), and among them include SUPRO® 500E, SUPRO® 620, SUPRO® 670, SUPRO® EX 33,l SUPRO® PLUS 2600F IP, SUPRO® PLUS 2640DS, SUPRO® PLUS 2800, SUPRO® PLUS 3000, and combinations thereof.

In another embodiment, the soy protein material may be a soy protein concentrate, which has a protein content of about 65% to less than about 90% on a moisture-free basis. Examples of suitable soy protein concentrates useful in the invention include the PROCON® product line, ALPHA® 12 and ALPHA® 5800, all of which are commercially available from Solae, LLC. Alternatively, soy protein concentrate may be blended with the soy protein isolate to substitute for a portion of the soy protein isolate as a source of soy protein material. Typically, if a soy protein concentrate is substituted for a portion of the soy protein isolate, the soy protein concentrate is substituted for up to about 40% of the soy protein isolate by weight, at most, and more preferably is substituted for up to about 30% of the soy protein isolate by weight.

In another iteration, the soy protein material may be soy flour, which has a protein content of about 49% to about 65% on a moisture-free basis. The soy flour may be defatted soy flour, partially defatted soy flour, or full fat soy flour. The soy flour may be blended with soy protein isolate or soy protein concentrate.

In still another iteration, the soy protein material may be material that has been separated into four major storage protein fractions or subunits (15S, 11S, 7S, and 2S) on the basis of sedimentation in a centrifuge. In general, the 11S fraction is highly enriched in glycinins, and the 7S fraction is highly enriched in beta-conglycinins.

In other embodiments, the protein material may be derived from a plant other than soy. By way of non-limiting example, suitable plants include amaranth, arrowroot, barley, buckwheat, canola, cassaya, channa (garbanzo), legumes, lentils, lupin, maize, millet, oat, pea, potato, rice, rye, sorghum, sunflower, tapioca, triticale, wheat, and mixtures thereof. Especially preferred plant proteins include barley, canola, lupin, maize, oat, pea, potato, rice, wheat, and combinations thereof. In one iteration, the plant protein material may be canola meal, canola protein isolate, canola protein concentrate, and combinations thereof. In another iteration, the plant protein material may be maize or corn protein powder, maize or corn protein concentrate, maize or corn protein isolate, maize or corn germ, maize or corn gluten, maize or corn gluten meal, maize or corn flour, zein protein, and combinations thereof. In still another iteration, the plant protein material may be barley powder, barley protein concentrate, barley protein isolate, barley meal, barley flour, and combinations thereof. In an alternate iteration, the plant protein material may be lupin flour, lupin protein isolate, lupin protein concentrate, and combinations thereof. In another alternate embodiment, the plant protein material may be oatmeal, oat flour, oat protein flour, oat protein isolate, oat protein concentrate, and combinations thereof. In yet another iteration, the plant protein material may be pea flour, pea protein isolate, pea protein concentrate, and combinations thereof. In still another iteration, the plant protein material may be potato protein powder, potato protein isolate, potato protein concentrate, potato flour, and combinations thereof. In a further embodiment, the plant protein material may be rice flour, rice meal, rice protein powder, rice protein isolate, rice protein concentrate, and combinations thereof. In another alternate iteration, the plant protein material may be wheat protein powder, wheat gluten, wheat germ, wheat flour, wheat protein isolate, wheat protein concentrate, solubilized wheat proteins, and combinations thereof.

In other embodiments, the protein material may be derived from an animal source. In one iteration, the animal protein material may be derived from eggs. Non-limiting examples of suitable egg proteins include powdered egg, dried egg solids, dried egg white protein, liquid egg white protein, egg white protein powder, isolated ovalbumin protein, and combinations thereof. Egg proteins may be derived from the eggs of chicken, duck, goose, quail, or other birds. In an alternate iteration, the protein material may be derived from a dairy source. Suitable dairy proteins include non-fat dry milk powder, milk protein isolate, milk protein concentrate, acid casein, caseinate (e.g., sodium caseinate, calcium caseinate, and the like), whey protein isolate, whey protein concentrate, and combinations thereof. The milk protein material may be derived from cows, goats, sheep, donkeys, camels, camelids, yaks, water buffalos, etc. In a further iteration, the protein may be derived from the muscles, organs, connective tissues, or skeletons of land-based or aquatic animals. As an example, the animal protein may be gelatin, which is produced by partial hydrolysis of collagen extracted from the bones, connective tissues, organs, etc, from cattle or other animals.

It is also envisioned that combinations of a soy protein material and at least one other protein material also may be used in the process of the invention. That is, a protein hydrolysate composition may be prepared from a combination of a soy protein material and at least one other protein material. In one embodiment, a protein hydrolysate composition may be prepared from a combination of a soy protein material and at least one other protein material selected from the group consisting of vegetable protein material, animal protein material, dairy protein material, egg protein material, and combinations thereof. The vegetable protein material can include barley, canola, lupin, maize, oat, pea, potato, rice, wheat, any other vegetable protein known in the art, and combinations thereof.

The concentrations of the soy protein material and the at least one other protein material used in combination can and will vary. The amount of soy protein material may range from about 1% to about 99% of the total protein used in the combination. In one embodiment, the amount of soy protein material may range from about 1% to about 20% of the total protein used in combination. In another embodiment, the amount of soy protein material may range from about 20% to about 40% of the total protein used in combination. In still another embodiment, the amount of soy protein material may range from about 40% to about 80% of the total protein used in combination. In a further embodiment, the amount of soy protein material may range from about 80% to about 99% of the total protein used in combination. Likewise, the amount of the at least one other protein material may range from about 1% to about 99% of the total protein used in combination. In one embodiment, the amount of the at least one other protein material may range from about 1% to about 20% of the total protein used in combination. In another embodiment, the amount of the at least one other protein material may range from about 20% to about 40% of the total protein used in combination. In still another embodiment, the amount of the at least one other protein material may range from about 40% to about 80% of the total protein used in combination. In a further embodiment, the amount of the at least one other protein material may range from about 80% to about 99% of the total protein used in combination.

In the process of the invention, the protein material is typically mixed or dispersed in water to form a slurry comprising about 1% to about 20% protein by weight (on an “as is” basis). In one embodiment, the slurry may comprise about 1% to about 5% protein (as is) by weight. In another embodiment, the slurry may comprise about 6% to about 10% protein (as is) by weight. In a further embodiment, the slurry may comprise about 11% to about 15% protein (as is) by weight. In still another embodiment, the slurry may comprise about 16% to about 20% protein (as is) by weight.

After the protein material is dispersed in water, the slurry of protein material may be heated from about 70° C. to about 90° C. for about 2 minutes to about 20 minutes to inactivate putative endogenous protease inhibitors. Typically, the pH and the temperature of the protein slurry are adjusted so as to optimize the hydrolysis reaction, and in particular, to ensure that the endopeptidase used in the hydrolysis reaction functions near its optimal activity level. The pH of the protein slurry may be adjusted and monitored according to methods generally known in the art. The pH of the protein slurry may be adjusted and maintained at a value from about pH 7.0 to about pH 11.0. In one embodiment, the pH of the protein slurry may be adjusted and maintained at from about pH 7.0 to about pH 8.0. In another embodiment, the pH of the protein slurry may be adjusted and maintained at from about pH 8.0 to about pH 9.0. In yet another embodiment, the pH of the protein slurry may be adjusted and maintained at from about pH 9.0 to about pH 10.0. In a preferred embodiment, the pH of the protein slurry may be adjusted and maintained at about pH 8.0 to about pH 8.5.

The temperature of the protein slurry is preferably adjusted and maintained at from about 30° C., preferably at least about 50° C. to about 80° C. during the hydrolysis reaction in accordance with methods known in the art. In general, temperatures above this range may inactivate the endopeptidase. Temperatures below this range tend to slow the activity of the endopeptidase. In a one embodiment, the temperature of the protein slurry may be adjusted and maintained at from about 50° C. to about 60° C. during the hydrolysis reaction. In another embodiment, the temperature of the protein slurry may be adjusted and maintained at from about 60° C. to about 70° C. during the hydrolysis reaction. In still another embodiment, the temperature of the protein slurry may be adjusted and maintained at from about 70° C. to about 80° C. during the hydrolysis reaction.

ii. Endopeptidase

The hydrolysis reaction generally is initiated by adding at least one endopeptidase to the slurry of protein material to form a reaction mixture. The endopeptidase catalyzes cleavage of peptides bonds within the proteins of the protein material to form a mixture of smaller sized oligopeptides. Endopeptidases are enzymes that generally cleave peptide bonds within the inner regions of a polypeptide chain.

Several endopeptidases are suitable for use in the process of the invention. In general, the endopeptidase will have a broad spectrum of activity (i.e., will hydrolyze the peptide bond between essentially any two amino acid residues). Typically, the endopeptidase may be a food-grade enzyme having optimal activity at a pH from about 7.0 to about 11.0 and at a temperature from about 30° C., preferably at least about 50° C. to about 80° C. Preferably, the endopeptidase will be an enzyme of microbial origin. The use of microbial enzymes, rather than animal or plant enzymes, is advantageous in that microbial enzymes exhibit a broad spectrum of characteristics (pH optima, temperature etc.) and may be consistently obtainable in relatively large quantities. In general, the endopeptidase will be a member of the serine peptidase family (see MEROPS Peptidase Database, release 8.00A; http//merops.sanger.ac.uk).

In one embodiment, the endopeptidase may be the subtilisin protease, or variant thereof, derived from Bacillus lichniformis (MEROPS Accession No. MER000309) that is available under the tradename ALCALASE® from Novozymes (Bagsvaerd, Denmark). In another embodiment, the endopeptidase may be a subtilisin, or variant thereof, derived from another microorganism. In a preferred embodiment, the endopeptidase may be serine protease (SP1) from Nocardiopsis prasina (International Patent Application No. WO02005035747, which is incorporated herein by reference in its entirety). The amino acid sequence of SP1 is ADIIGGLAYT MGGRCSVGFA ATNAAGQPGF VTAGHCGRVG TQVTIGNGRG VFEQSVFPGN DAAFVRGTSN FTLTNLVSRY NTGGYATVAG HNQAPIGSSV CRSGSTTGWH CGTIQARGQS VSYPEGTVTN MTRTTVCAEP GDSGGSYISG TQAQGVTSGG SGNCRTGGTT FYQEVTPMVN SWGVRLRT (SEQ ID NO:1).

In a further embodiment, the endopeptidase may be a serine protease having an amino acid sequence that is at least 80%, 81%, 82%, 83%, 84%, or 85% identical to SEQ ID NO:1 or a fragment thereof. In another embodiment, the endopeptidase may be a serine protease having an amino acid sequence that is at least 86%, 87%, 88%, 89%, 90%, 91%, or 92% identical to SEQ ID NO:1 or a fragment thereof. In still another embodiment, the endopeptidase may be a serine protease having an amino acid sequence that is at least 93%, 94%, 95%, 96%, 97%, 98% or 99% to SEQ ID NO:1 or a fragment thereof.

For purposes of the present invention, the alignment of two amino acid sequences may be determined by using the Needle program from the EMBOSS package (Rice, P., Longden, I. and Bleasby, A. (2000) EMBOSS: The European Molecular Biology Open Software Suite. Trends in Genetics 16, (6) pp 276-277; http://emboss.org) version 2.8.0. The Needle program implements the global alignment algorithm described in Needleman, S. B. and Wunsch, C. D. (1970) J. Mol. Biol. 48, 443-453. The substitution matrix used is BLOSUM62, gap opening penalty is 10, and gap extension penalty is 0.5. In general, the percentage of sequence identity is determined by comparing two optimally aligned sequences over a comparison window, wherein the portion of the amino acid sequence in the comparison window may comprise additions or deletions (i.e., gaps) as compared to the reference sequence (which does not comprise additions or deletions) for optimal alignment of the two sequences. The percentage is calculated by determining the number of positions at which an identical amino acid occurs in both sequences to yield the number of matched positions, dividing the number of matched positions by the total number of positions in the shortest of the two sequences in the window of comparison, and multiplying the result by 100 to yield the percentage of sequence identity.

A skilled practitioner will understand that an amino acid residue may be substituted with another amino acid residue having a similar side chain without affecting the function of the polypeptide. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acid substitution groups include: valine-leucine-isoleucine, phenylalanine-tyrosine, lysine-arginine, alanine-valine, and asparagine-glutamine. Thus, the endopeptidase may have at least one conservative amino acid substitutions with respect to SEQ ID NO:1. In one embodiment, the endopeptidase may have about 45 conservative amino acid substitutions with respect to SEQ ID NO:1. In another embodiment, the endopeptidase may have about 35 conservative amino acid substitutions with respect to SEQ ID NO:1. In still embodiment, the endopeptidase may have about 25 conservative amino acid substitutions with respect to SEQ ID NO:1. In yet another embodiment, the endopeptidase may have about 15 conservative amino acid substitutions with respect to SEQ ID NO:1. In an alternate embodiment, the endopeptidase may have about 10 conservative amino acid substitutions with respect to SEQ ID NO:1. In yet another embodiment, the endopeptidase may have about 5 conservative amino acid substitutions with respect to SEQ ID NO:1. In a further embodiment, the endopeptidase may have about one conservative amino acid substitutions with respect to SEQ ID NO:1.

It is also envisioned that combinations of endopeptidases may also be utilize in the process of the invention. For example, the protein material may be contacted with a mixture of ALCALASE® and SP1. Alternatively, the protein material may be contacted with a mixture of SP1 and an endopeptidase that is at least 80% identical to SEQ ID No:1. Similarly, other combinations of broad spectrum serine proteases may be used without departing from the scope of the invention.

Exemplary combinations of protein material and endopeptidase(s) are presented in Table A.

TABLE A
Preferred Combinations.

| Protein Material | Endopeptidase |
|---|---|
| Soy | SP1 |
| Soy | ALCALASE ® |
| Soy | SP1 and ALCALASE ® |
| Barley | SP1 |
| Barley | ALCALASE ® |
| Barley | SP1 and ALCALASE ® |
| Canola | SP1 |
| Canola | ALCALASE ® |
| Canola | SP1 and ALCALASE ® |
| Lupin | SP1 |
| Lupin | ALCALASE ® |
| Lupin | SP1 and ALCALASE ® |
| Maize | SP1 |
| Maize | ALCALASE ® |
| Maize | SP1 and ALCALASE ® |
| Oat | SP1 |
| Oat | ALCALASE ® |
| Oat | SP1 and ALCALASE ® |
| Pea | SP1 |
| Pea | ALCALASE ® |
| Pea | SP1 and ALCALASE ® |
| Potato | SP1 |
| Potato | ALCALASE ® |
| Potato | SP1 and ALCALASE ® |
| Rice | SP1 |
| Rice | ALCALASE ® |
| Rice | SP1 and ALCALASE ® |
| Wheat | SP1 |
| Wheat | ALCALASE ® |
| Wheat | SP1 and ALCALASE ® |
| Egg | SP1 |
| Egg | ALCALASE ® |
| Egg | SP1 and ALCALASE ® |
| Dairy | SP1 |
| Dairy | ALCALASE ® |
| Dairy | SP1 and ALCALASE ® |
| Animal (e.g., gelatin) | SP1 |
| Animal (e.g., gelatin) | ALCALASE ® |
| Animal (e.g., gelatin) | SP1 and ALCALASE ® |
| Soy and Barley | SP1 |
| Soy and Barley | ALCALASE ® |
| Soy and Barley | SP1 and ALCALASE ® |
| Soy and Canola | SP1 |
| Soy and Canola | ALCALASE ® |
| Soy and Canola | SP1 and ALCALASE ® |
| Soy and Lupin | SP1 |
| Soy and Lupin | ALCALASE ® |
| Soy and Lupin | SP1 and ALCALASE ® |
| Soy and Maize | SP1 |
| Soy and Maize | ALCALASE ® |
| Soy and Maize | SP1 and ALCALASE ® |
| Soy and Oat | SP1 |
| Soy and Oat | ALCALASE ® |
| Soy and Oat | SP1 and ALCALASE ® |
| Soy and Pea | SP1 |
| Soy and Pea | ALCALASE ® |
| Soy and Pea | SP1 and ALCALASE ® |
| Soy and Potato | SP1 |
| Soy and Potato | ALCALASE ® |
| Soy and Potato | SP1 and ALCALASE ® |
| Soy and Rice | SP1 |
| Soy and Rice | ALCALASE ® |
| Soy and Rice | SP1 and ALCALASE ® |
| Soy and Wheat | SP1 |
| Soy and Wheat | ALCALASE ® |
| Soy and Wheat | SP1 and ALCALASE ® |
| Soy and Egg | SP1 |
| Soy and Egg | ALCALASE ® |
| Soy and Egg | SP1 and ALCALASE ® |
| Soy and Dairy | SP1 |
| Soy and Dairy | ALCALASE ® |
| Soy and Dairy | SP1 and ALCALASE ® |
| Soy and Animal (e.g., gelatin) | SP1 |
| Soy and Animal (e.g., gelatin) | ALCALASE ® |
| Soy and Animal (e.g., gelatin) | SP1 and ALCALASE ® |

The amount of endopeptidase added to the protein slurry can and will vary depending upon the protein material, the desired degree of hydrolysis, and the duration of the hydrolysis reaction. In general, the amount of endopeptidase will range from about 1 mg of enzyme protein to about 5000 mg of enzyme protein per kilogram of starting protein material. In another embodiment, the amount of endopeptidase may range from 50 mg of enzyme protein to about 1000 mg of enzyme protein per kilogram of starting protein material. In yet another embodiment, the amount of endopeptidase may range from about 1000 mg of enzyme protein to about 5000 mg of enzyme protein per kilogram of starting protein material.

As will be appreciated by a skilled artisan, the duration of the hydrolysis reaction can and will vary, depending upon the concentration of the endopeptidase and the desired degree of hydrolysis, for example. Generally speaking, the duration of the hydrolysis reaction may range from a few minutes to many hours, such as, from about 5 minutes to about 48 hours. In a preferred embodiment, the duration of the reaction may be about 30 minutes to about 120 minutes.

To terminate the hydrolysis reaction, the reaction mixture may be heated to a temperature that is high enough to inactivate the endopeptidase. For example, heating the reaction mixture to a temperature of approximately 90° C. will substantially heat-inactivate the most proteases. Alternatively, the hydrolysis reaction may be terminated by lowering the pH of the reaction mixture to about 4.0 and heating the reaction mix to a temperature greater than about 80° C. Examples of acids that may be used to lower the pH of the reaction mixture include citric acid, formic acid, fumaric acid, hydrochloric acid, lactic acid, malic acid, phosphoric acid, and combinations thereof.

b. Lowering the pH of the Hydrolysate

The second step of the process comprises lowering the pH of the protein hydrolysate to a value less than about pH 7.0. In one embodiment, the pH of the protein hydrolysate may be adjusted to a level from about pH 6.0 to about pH 7.0. In another embodiment, the pH of the protein hydrolysate may be adjusted to a level from about pH 5.0 to about pH 6.0. In further embodiment, the pH of the protein hydrolysate may be adjusted to a level from about pH 4.0 to about pH 5.0. In still another embodiment, the pH of the protein hydrolysate may be adjusted to a level from about pH 3.0 to about pH 4.0. In another alternate embodiment, the pH of the protein hydrolysate may be adjusted to a level from about pH 2.0 to about pH 3.0. In still another alternate embodiment, the pH of the protein hydrolysate may be adjusted to a level from about pH 1.0 to about pH 2.0. In preferred embodiments, the pH of the protein hydrolysate may be adjusted to a pH value of less than about pH 5.0.

In general, an acidic solution will be used to adjust the pH level of the protein hydrolysate. Non-limiting examples of acids that may be used to adjust the pH of they hydrolysate include citric acid, formic acid, fumaric acid, hydrochloric acid, lactic acid, malic acid, phosphoric acid and combinations thereof.

### II. Protein Hydrolysate Compositions

Another aspect of the invention encompasses a protein hydrolysate composition comprising a mixture of oligopeptides having an average size of less than about 10,000 Daltons. Additionally, the composition has a degree of hydrolysis of at least about 2.5%, generally at least about 5.0%, preferably at least about 7.5%, and most preferably at least about 10% and a solid solubility index of at least about 60% at a pH of less than about 7.0.

The degree of hydrolysis (% DH) refers to the percentage of peptide bonds cleaved versus the starting number of total peptide bonds. For example, if an intact protein containing five hundred total peptide bonds is hydrolyzed until fifty of the peptide bonds are cleaved, then the degree of hydrolysis of the resulting hydrolysate is 10%. The degree of hydrolysis may be determined using the o-phthaldialdehye (OPA) method or the trinitrobenzene sulfonic (TNBS) colorimetric method, as detailed in the examples. The higher the degree of hydrolysis the greater the extent of protein hydrolysis. Typically, as the protein is further hydrolyzed (i.e., the higher the degree of hydrolysis), the molecular weight of the peptide fragments decreases, the peptide profile changes accordingly, and the viscosity of the mixture decreases. The degree of hydrolysis may be measured in the entire hydrolysate (i.e., whole fraction) or the degree of hydrolysis may be measured in the soluble fraction of the hydrolysate (i.e., the supernatant fraction after centrifugation of the hydrolysate at about 500-1000×g for about 5-10 min).

The degree of hydrolysis of the protein hydrolysate composition can and will vary depending upon the source of the protein material, the endopeptidase(s) used, and the conditions of the hydrolysis reaction. In general, the degree of hydrolysis of the protein hydrolysate composition will be greater than about 10%. In one embodiment, the degree of hydrolysis of the protein hydrolysate composition may range from about 10% to about 15%. In another embodiment, the degree of hydrolysis of the protein hydrolysate composition may range from about 15% to about 20%. In a further embodiment, the degree of hydrolysis of the protein hydrolysate composition may range from about 20% to about 25%. In still another embodiment, the degree of hydrolysis of the protein hydrolysate composition may range from about 25% to about 35%.

The solid solubility index (SSI) or percent of soluble solids is a measure of the solubility of the solids (i.e., polypeptides and fragments thereof) comprising a protein hydrolysate composition. The amount of soluble solids may be estimated by measuring the amount of solids in solution before and after centrifugation (e.g., about 500-1000×g for about 5-10 min). Alternatively, the amount of soluble solids may be determined by estimating the amount of protein in the composition before and after centrifugation using a technique well known in the art (such as, e.g., a bicinchoninic acid (BCA) protein determination colorimetric assay).

In general, the protein hydrolysate compositions of the invention will have a solid solubility index of at least 60% at a pH value of less than about pH 7.0. In one embodiment, the solid solubility index of the hydrolysate may range from about 60% to about 70% at a pH value of less than about pH 7.0. In another embodiment, the solid solubility index of the hydrolysate may range from about 70% to about 80% at a pH value of less than about pH 7.0. In still another embodiment, the solid solubility index of the hydrolysate may range from about 80% to about 90% at a pH value of less than about pH 7.0. In yet another embodiment, the solid solubility index of the hydrolysate may range from about 90% to about 99% at a pH value of less than about pH 7.0.

In general, the protein hydrolysate composition, as compared to the starting protein material, will comprise a mixture of oligopeptides of varying lengths and molecular sizes. The molecular sizes of the oligopeptides may range from about 75 Daltons (i.e., free glycine) to about 100,000 Daltons. In general, the average size of the oligopeptides forming the protein hydrolysate composition will be less than about 10,000 Daltons. In one embodiment, the average size of the oligopeptides forming the protein hydrolysate composition may be less than about 8000 Daltons. In another embodiment, the average size of the oligopeptides forming the protein hydrolysate composition may be less than about 6000 Daltons. In a further embodiment, the average size of the oligopeptides forming the protein hydrolysate composition may be less than about 4000 Daltons. In an alternate embodiment, the average size of the oligopeptides forming the protein hydrolysate composition may be less than about 2000 Daltons. In yet another embodiment, the average size of the oligopeptides forming the protein hydrolysate composition may be less than about 1000 Daltons.

The protein hydrolysate compositions of the invention generally are substantially stable. As used herein, “stability” refers to the lack of sediment formation over time. The protein hydrolysate compositions may be stored at room temperature (i.e., about 23° C.) or a refrigerated temperature (i.e., about 4° C.). In one embodiment, the protein hydrolysate composition may be stable for about one week to about four weeks. In another embodiment, the protein hydrolysate composition may be stable for about one month to about six months. In a further embodiment, the protein hydrolysate composition may be stable for more than about six months.

Moreover, the protein hydrolysate compositions of the invention may be dried. For example the protein hydrolysate composition may be spray dried. The temperature of the spray dryer inlet may range from about 260° C. (500° F.) to about 316° C. (600° F.) and the exhaust temperature may range from about 82° C. (180° F.) to about 38° C. (100° F.). Alternatively, the protein hydrolysate composition may be vacuum dried, freeze dried, or dried using other procedures known in the art.

In embodiments in which the protein material is soy, the protein hydrolysate composition may comprise at least one oligopeptide having an amino acid sequence that corresponds to or is derived from the group consisting of SEQ ID NO:2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, and 45. In one embodiment, the protein hydrolysate composition may comprise at least ten oligopeptides or fragments thereof selected from the group consisting of SEQ ID NO:2-45. In another embodiment, the protein hydrolysate composition may comprise at least 20 oligopeptides or fragments thereof selected from the group consisting of SEQ ID NO:2-45. In a further embodiment, the protein hydrolysate composition may comprise at least 30 oligopeptides or fragments thereof selected from the group consisting of SEQ ID NO:2-45. In yet another embodiment, the protein hydrolysate composition may comprise at least 40 oligopeptides or fragments thereof selected from the group consisting of SEQ ID NO:2-45. In an alternate embodiment, the protein hydrolysate composition may comprise oligopeptides or fragments thereof corresponding to SEQ ID NO:2-45.

The invention also encompasses any of the oligopeptides identified in the soy protein hydrolysates. For example, an oligopeptide may be purified by a chromatographic method, such as size exclusion chromatography, ion exchange chromatography, affinity chromatography, hydrophobic interaction chromatography, reverse phase chromatography, and the like. Alternatively, an oligopeptide may be synthesized, using a synthesis method known to those of skill in the art.

The protein hydrolysate compositions, and in particular the soy protein hydrolysate compositions of the invention, may have enhanced sensory and taste profiles with respect to the starting protein material or other hydrolysate compositions. In addition to the number of polypeptide fragments formed and their respective sizes, the degree of hydrolysis typically impacts other physical properties and sensory properties of the resulting soy protein hydrolysate composition. In general, the soy protein hydrolysate compositions of the invention have substantially less bitter sensory attributes and improved overall liking scores than commercially available soy protein hydrolysates.

### III. Food Products Comprising a Protein Hydrolysate Composition

A further aspect of the present invention is the provision of a food product comprising any of the protein hydrolysate compositions described herein. Alternatively, the food product may comprise any of the isolated polypeptides, or fragments thereof, described herein.

The selection of a particular protein hydrolysate composition will vary depending upon the desired food or beverage product. In some embodiments, the protein hydrolysate composition may be derived from soy protein. In other embodiments, the protein hydrolysate composition may be derived from vegetable protein material, animal protein material, dairy protein material, egg protein material, and combinations thereof. The vegetable protein material can include barley, canola, lupin, maize, oat, pea, potato, rice, wheat, any other vegetable protein known in the art, and combinations thereof. In still other embodiments, the protein hydrolysate composition may be derived from a combination of soy and at least one other protein source selected from the group consisting of vegetable protein material, animal protein material, dairy protein material, egg protein material, and combinations thereof. The vegetable protein material can include barley, canola, lupin, maize, oat, pea, potato, rice, wheat, any other vegetable protein known in the art, and combinations thereof. In alternate embodiments, the protein hydrolysate composition may comprise a combination of different protein hydrolysates. In additional embodiments, the protein hydrolysate composition may comprise isolated or synthetic polypeptides selected from the group of amino acid sequences consisting of SEQ ID NO:2-45. The degree of hydrolysis of the protein hydrolysate composition used to make a food product also will vary depending upon, for example, the source of the protein material and the desired food product.

The food or beverage product can further include an edible material. The selection of the appropriate edible material will vary depending on the desired food or beverage product. The edible material may be a plant-derived material, an animal-derived material, or a biomaterial (i.e., a protein, a carbohydrate, a lipid, etc.) isolated from a plant-derived material, an animal-derived material, and so forth.

The beverage may be a ready-to-drink (RTD) beverage. The beverage may be a substantially clear beverage such as a juice beverage, a fruit flavored beverage, a carbonated beverage, a sports drink, a nutritional supplement beverage, a weight management beverage, or an alcohol-based fruit beverage. Such substantially clear beverages typically have pH values of less than about pH 5.0. In a preferred embodiment, the substantially clear beverage may have a pH value of less than about pH 4.0.

Alternatively, the beverage may be a substantially cloudy beverage such as a meal replacement drink, a protein shake, a coffee-based beverage, a nutritional supplement beverage, or a weight management beverage. In general, the substantially cloudy beverage will have a pH value of less than about pH 7.0.

In embodiments in which the product is a beverage, the edible material may include fruit juice, sugar, milk, non-fat dry milk powder, caseinate, soy protein concentrate, soy protein isolate, whey protein concentrate, whey protein isolate, isolated milk protein, chocolate, cocoa powder, coffee, tea, and combinations thereof. The beverage may further comprise sweetening agents (such as glucose, sucrose, fructose, maltodextrin, sucralose, corn syrup, honey, maple syrup, etc.), flavoring agents (e.g., fruit flavors, chocolate flavor, vanilla flavor, etc), emulsifying or thickening agents (e.g., lecithin, carrageenan, cellulose gum, cellulose gel, starch, gum arabic, xanthan gum, and the like); stabilizing agents, lipid materials (e.g., canola oil, sunflower oil, high oleic sunflower oil, fat powder, etc.), preservatives (e.g., potassium sorbate, sorbic acid, and so forth), antioxidants (e.g., ascorbic acid, sodium ascorbate, etc.), coloring agents, vitamins, minerals, and combinations thereof.

In an alternate embodiment, the food product may be a food bar, such as a granola bar, a snack bar, a cereal bar, as a breakfast bar, a nutrition bar, an energy bar, or a weight management bar.

### DEFINITIONS

To facilitate understanding of the invention, several terms are defined below.

The term “degree of hydrolysis” refers to the percentage of the total number of peptide bonds that are cleaved.

The term “endopeptidase” refers to an enzyme that hydrolyzes internal peptide bonds in oligopeptide or polypeptide chains. The group of endopeptidases comprises enzyme subclasses EC 3.4.21-25 (International Union of Biochemistry and Molecular Biology enzyme classification system).

A “food grade enzyme” is an enzyme that is generally recognized as safe (GRAS) approved and is safe when consumed by an organism, such as a human. Typically, the enzyme and the product from which the enzyme may be derived are produced in accordance with applicable legal and regulatory guidelines.

A “hydrolysate” is a reaction product obtained when a compound is cleaved through the effect of water. Protein hydrolysates occur subsequent to thermal, chemical, or enzymatic degradation. During the reaction, large molecules are broken into smaller proteins, soluble proteins, oligopeptides, peptide fragments, and free amino acids.

The term “polypeptide” encompasses oligopeptides.

The term “sensory attribute,” such as used to describe terms like “bitter,” “grain,” or “astringent” is determined in accordance with the SQS Scoring System as specifically delineated in Example 2.

The term “solid solubility index” refers to the percentage of soluble proteins or soluble solids.

The terms “soy protein isolate” or “isolated soy protein,” as used herein, refer to a soy material having a protein content of at least about 90% soy protein on a moisture free basis. A soy protein isolate is formed from soybeans by removing the hull and germ of the soybean from the cotyledon, flaking or grinding the cotyledon and removing oil from the flaked or ground cotyledon, separating the soy protein and carbohydrates of the cotyledon from the cotyledon fiber, and subsequently separating the soy protein from the carbohydrates.

The term “soy protein concentrate” as used herein is a soy material having a protein content of from about 65% to less than about 90% soy protein on a moisture-free basis. Soy protein concentrate also contains soy cotyledon fiber, typically from about 3.5% up to about 20% soy cotyledon fiber by weight on a moisture-free basis. A soy protein concentrate is formed from soybeans by removing the hull and germ of the soybean, flaking or grinding the cotyledon and removing oil from the flaked or ground cotyledon, and separating the soy protein and soy cotyledon fiber from the soluble carbohydrates of the cotyledon.

The term “soy flour” as used herein, refers to a comminuted form of defatted, partially defatted, or full fat soybean material having a size such that the particles can pass through a No. 100 mesh (U.S. Standard) screen. The soy cake, chips, flakes, meal, or mixture of the materials are comminuted into soy flour using conventional soy grinding processes. Soy flour has a soy protein content of about 49% to about 65% on a moisture free basis. Preferably the flour is very finely ground, most preferably so that less than about 1% of the flour is retained on a 300 mesh (U.S. Standard) screen.

The term “soy cotyledon fiber” as used herein refers to the polysaccharide portion of soy cotyledons containing at least about 70% dietary fiber. Soy cotyledon fiber typically contains some minor amounts of soy protein, but may also be 100% fiber. Soy cotyledon fiber, as used herein, does not refer to, or include, soy hull fiber. Generally, soy cotyledon fiber is formed from soybeans by removing the hull and germ of the soybean, flaking or grinding the cotyledon and removing oil from the flaked or ground cotyledon, and separating the soy cotyledon fiber from the soy material and carbohydrates of the cotyledon.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples illustrate various embodiments of the invention.

### Example 1

### Hydrolysis of Soy Protein with SP1 or ALCALASE®

The following study was undertaken to determine whether hydrolysis of soy protein with different endopeptidases could increase the solubility of the hydrolysate at acid pH (i.e., near its isoelectric point)

Hydrolysis Reactions. The starting material was 10% soy protein isolate (e.g., SUPRO® 500E) suspended in 0.1 M sodium phosphate, pH 8.5. The pH of the protein slurry was adjusted to 8.0 with HCl. The protein slurry was heated to about 70° C. and the mixture was hydrolyzed with either serine protease (SP1) from Nocardiopsis prasina or subtilisin (ALCALASE®) from Bacillus licheniformis. Each endopeptidase was used at a concentration of 19.5 mg, 39.0 mg, 78.1 mg, 156.3 mg, or 312.5 mg of protease per kg of soy protein isolate. The hydrolysis reaction was allowed to proceed at 70° C. for 120 minutes. The reaction was stopped with the addition of 1 M sodium formate, pH 3.7, such that the pH of the mixture was about 4.0 and the final concentration of soy protein in the hydrolysate was 5%.

Solubility Analysis. The percent of soluble solids or the solid solubility index (SSI) of each of the resultant hydrolysates was determined by measuring the soluble protein using a bicinchoninic acid (BCA) based protein assay (e.g., a Micro BCA™ Protein Assay Kit; Sigma-Aldrich, St. Louis, Mo.). For this, each hydrolysate was centrifuged at 500×g for 10 min to precipitate any insoluble fragments. Each supernatant fraction can be diluted with different concentration (i.e., 10-, 20-, 40-, and 80-fold with distilled H₂O). In this case 10 fold dilution was used. A 20 μL aliquot of each dilution was transferred to a microtiter plate and 160 μL of BCA working reagent was added. After 30 minutes of incubation at 37° C., the absorbance at 562 nm was measured. A BSA standard dilution (0-1 mg/mL) curve was also run. The positive control was a commercially available soy protein hydrolysate (i.e., HXP114). Solubility was calculated assuming that the positive control was 100% soluble and is expressed as percent (i.e., % SSI).

The percent of soluble solids in each of the hydrolysates is presented in Table 1. At each endopeptidase concentration, the SP1 hydrolysates had increased solubility relative to that of the ALCALASE® hydrolysates.

TABLE 1
Solubility of Hydrolysates.

Solid Solubility Index (%)

| Protease concentration | ALCALASE ® | SP1 |
|---|---|---|
| (mg/kg soy) | (% ± s.d.) | (% ± s.d.) |
| 312.5 | 72.7 ± 0.6 | 85.1 ± 0.1 |
| 156.3 | 69.1 ± 1.7 | 84.6 ± 2.4 |
| 78.1 | 60.7 ± 5.5 | 70.7 ± 13.6 |
| 39.0 | 63.5 ± 1.7 | 77 ± 3.3 |
| 19.5 | 58.5 ± 1.3 | 72.6 ± 4.2 |

Degree of hydrolysis. The degree of hydrolysis (% DH) of each of the hydrolysates was determined using the o-phthaldialdehyde (OPA) assay. For this, each hydrolysate (and non-hydrolyzed starting material) was diluted 50-fold with water. A 20 μL aliquot of each was mixed with 180 μL of OPA reagent (4 mM disodium tetraborate, 0.1% SDS, 0.24 mM OPA, 0.24 mM DTT in a well of a microtiter plate. Absorbance at 340 nm was measured. A standard curve with L-serine (0-0.5 mg/mL) was also included. The degree of hydrolysis was calculated by subtracting the % DH value of the non-hydrolyzed starting material from the % DH value of each hydrolysate.

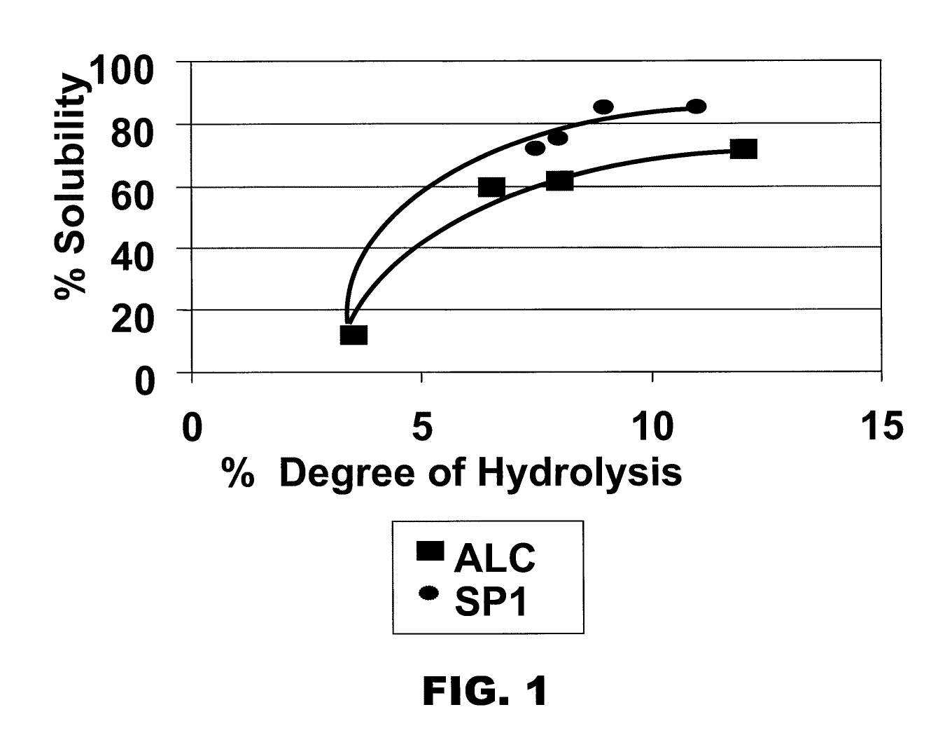

Table 2 presents the results. The degree of hydrolysis of each hydrolysate was similar at each concentration of protease. Furthermore, as shown in FIG. 1, the percent of soluble solids increased as the degree of hydrolysis increased for the different hydrolysates, indicating that these bench studies are predictive of the yields of soluble peptides.

TABLE 2
Degree of Hydrolysis.

| | |
|---|---|
| Protease | Degree of Hydrolysis (% ± s.d.) |

| concentration | ALCALASE ® | SP1 |
|---|---|---|
| (mg/kg soy) | Hydrolysate | Hydrolysate |
| 312.5 | 8.2 ± 0.1 | 7.2 ± 0.7 |
| 156.3 | 5.8 ± 0.5 | 5.8 ± 0.6 |
| 78.1 | 4.5 ± 1.2 | 3.8 ± 1.2 |
| 39.0 | 4.6 ± 0.6 | 4.8 ± 1.0 |
| 19.5 | 3.4 ± 0.3 | 4.2 ± 0.7 |

### Example 2

### SQS and Bitterness Analysis of SP1 and ALCALASE® Hydrolysates

The flavor profile of SP1 hydrolysates was compared to that of ALCALASE® hydrolysates. The two preparations were tested with respect to bitterness using the Solae Qualitative Screening (SQS) test. Hydrolysates were prepared essentially as described in Example 1, except that only one concentration of endopeptidase was used (i.e., 300 mg of protease/kg of soy) and the reaction was carried out at 60° C. for 120 min. The enzymes were inactivated by heating the hydrolysates to 85° C. for 15 min. The degree of hydrolysis and percent of soluble solids were determined essentially as described in Example 1.

Table 3 presents the degree of hydrolysis and % soluble solids (SSI) for each hydrolysate.

TABLE 3
Properties of Hydrolysates.

| Sample | % DH | SSI % |
|---|---|---|
| ALCALASE ® Hydrolysate | 11.4% | 74% |
| SP1 Hydrolysate | 11.6% | 86% |

The SQS method is based upon a direct comparison between a test sample and a control sample, and it provides both qualitative and directional quantitative differences. The control sample was a 5% slurry of untreated isolated soy protein. A panel of five to ten assessors was provided with aliquots of each test (diluted to a 5% slurry) and control sample. The samples were allowed equilibrate to room temperature before scoring.

The evaluation protocol comprised swirling a cup three times, while keeping the bottom of the cup on the table. After the sample sat for 2 seconds, each taster sipped about 10 mL (2 tsp), swished it about her/his mouth for 10 seconds, and then expectorated. The taster then rated the differences between the test sample and the control sample according to the scale presented in Table 4.

TABLE 4
SQS Scoring System

| SQS Score | Scale | Definition |
|---|---|---|
| 5 | Match | The test sample has virtually identical sensory |
| | | characteristics to the control sample by appearance, aroma, |
| | | flavor and texture. |
| 4 | Slight difference | The test sample has one or multiple ‘slight’ differences from |
| | | the control sample. These differences might not be noticed if |
| | | not in a side-by-side comparison with the control. |
| 3 | Moderate | The test sample has one or multiple ‘moderate’ differences |
| | difference | from the control sample. These differences would be |
| | | noticeable in a side-by-side comparison of the two samples |
| | | after one tasting of each. |
| 2 | Extreme | The test sample has one or multiple ‘extreme’ differences |
| | difference | from the control sample. These differences would be noticed |
| | | even if not in a side-by-side comparison. |
| 1 | Reject | The test sample has obvious defects that make it different |
| | | from the control sample. |

The SQS scores are presented in Table 5. In general, the SP1 hydrolysates had higher SQS scores than the ALCALASE® hydrolysates.

TABLE 5
SQS Scores

| Sample | Trial 1 | Trial 2 |
|---|---|---|
| Control | 5 (match) | 5 |
| ALCALASE ® Hydrolysate | 2.5 | 3.1 |
| SP1 Hydrolysate | 2.8 | 3.5 |

Each test sample was further evaluated to provide diagnostic information on how the test sample differed from the control sample with respect to bitterness. That is, if the test sample had slightly more, moderately more, or extremely more bitterness than the control sample, then a score of +1, +2, +3, respectively, was assigned. Likewise, if the test sample had slightly less, moderately less, or extremely less bitterness than the control sample, then a score of −1, −2, −3, respectively, was assigned. This analysis provided an assessment of the directional quantitative differences between the test sample and the control sample. If the test sample had no difference from the control, a score of zero (0) was assigned.

Table 6 presents the bitterness scores of the two hydrolysates used in two different trials. The SP1 hydrolysates were rated as being less bitter than that of the ALCALASE® hydrolysates in each trial.

TABLE 6
Bitterness scores.

| Sample | Trial 1 | Trial 2 |
|---|---|---|
| Control | 0 | 0 |
| ALCALASE ® Hydrolysate | 2.2 | 1.7 |
| SP1 Hydrolysate | 1.3 | 0.6 |

### Example 3

### Yield of SP1 and ALCALASE® Hydrolysates

Soy protein was hydrolyzed with either SP1 or ALCALASE® (ALC) (expressed as % enzyme protein) essentially as described in Example 1. The hydrolysis reactions were conducted at pH 8.0-8.5, at 60° C., for 30-60 min. The percent of soluble solids was determined essentially by the percent solids of the soluble over the whole fractions. Yield was calculated as the protein material concentration at the isoelectric point of pH 4.5. Yield is defined as a ratio of the percent of total solids to be centrifuged vs. percent of the solids in the soluble fraction after centrifugation.

The degree of hydrolysis was determined using a simplified trinitrobenzenesulfonic acid (TNBS) method (i.e., based on that of Adler-Nissen, 1979, J. Agric. Food Chem. 27(6):1256-1262). For this, 0.1 g of the soy protein hydrolysate was dissolved in 100 mL of 0.025 N NaOH. An aliquot (2.0 mL) of the hydrolysate solution was mixed with 8 mL of 0.05 M sodium borate buffer (pH 9.5). Two mL of the buffered hydrolysate solution was treated with 0.20 mL of 10% trinitrobenzene sulfonic acid, followed by incubation in the dark for 15 minutes at room temperature. The reaction was quenched by adding 4 mL of a 0.1 M sodium sulfite-0.1 M sodium phosphate solution (1:99 ratio), and the absorbance was read at 420 nm. A 0.1 mM glycine solution was used as the standard. The following calculation was used to determine the percent recovery for the glycine standard solution: (absorbance of glycine at 420 nm−absorbance of blank at 420 nm)×(100/0.710). Values of 94% or higher were considered acceptable.

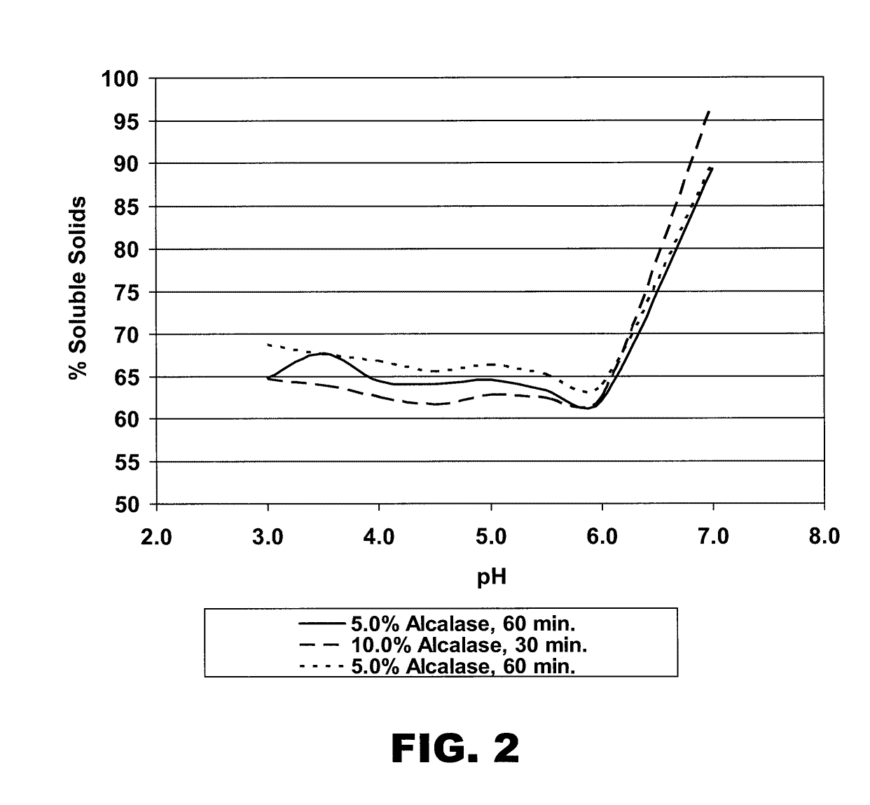

Table 7 presents the percent yield by solids, the percent soluble solids, and the degree of hydrolysis. It was found that hydrolysates prepared with either SP1 or ALC had similar yields at equal enzyme doses or similar degrees of hydrolysis. Furthermore, these data reveal that increased degrees of hydrolysis or levels of enzyme increased the yield. As shown in FIG. 2, hydrolysates with different degrees of hydrolysis were equally soluble at all pH levels.

TABLE 7
Yield Results

| | | Degree of |
|---|---|---|
| Sample | Yield | Hydrolysis |
| (% Enzyme Protein) | (%) | (%) |
| SP1 (0.030%) | 30.1 | 7.9 |
| SP1 (0.150%) | 58.0 | 12.6 |
| SP1 (0.150%) | 65.7 | 12.3 |
| SP1 (0.210%) | 81.4 | 13.3 |
| ALC (0.038%) | 31.5 | 11.0 |
| ALC (0.417%) | 63.9 | 11.1 |
| ALC (0.125%) | 48.2 | 12.2 |
| ALC (0.208%) | 67.8 | 17.3 |
| ALC (0.208%) | 75.4 | 17.9 |
| ALC (0.417%) | 79.2 | 16.4 |
| ALC (0.208%) | 70.1 | 18.5 |
| ALC (0.208%) | 63.3 | 17.7 |

### Example 4

### Sensory Analysis of SP1 and ALCALASE® Hydrolysates

Complete flavor profiles of SP1 and ALC soy hydrolysates, prepared essentially as described in Example 3, were also evaluated. The hydrolysates were scored with respect to astringency, bitterness, saltiness, and other attributes as compared to a control sample.

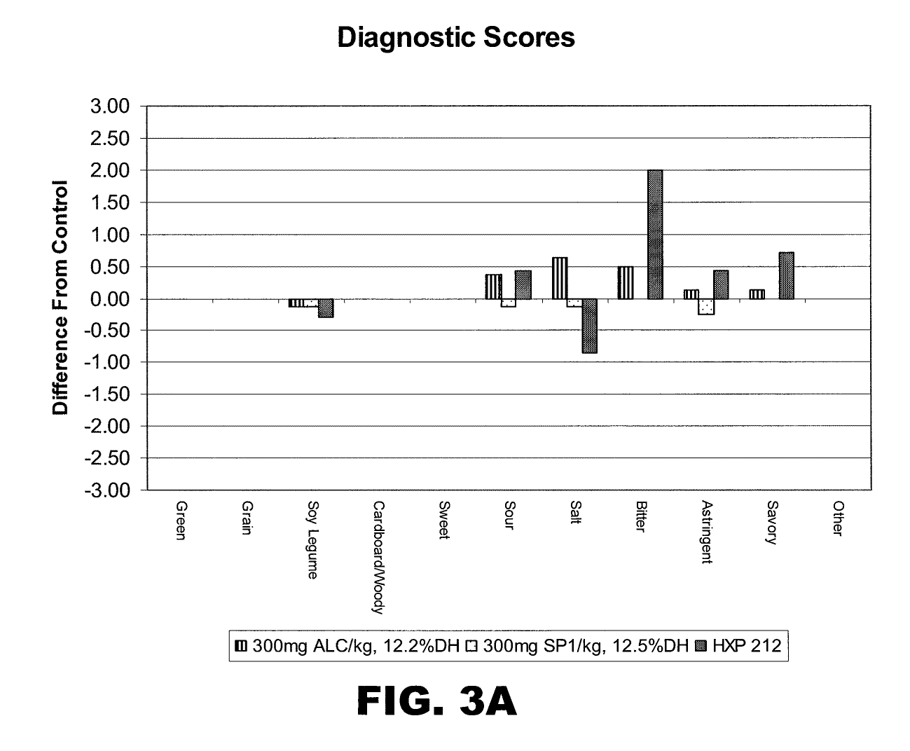

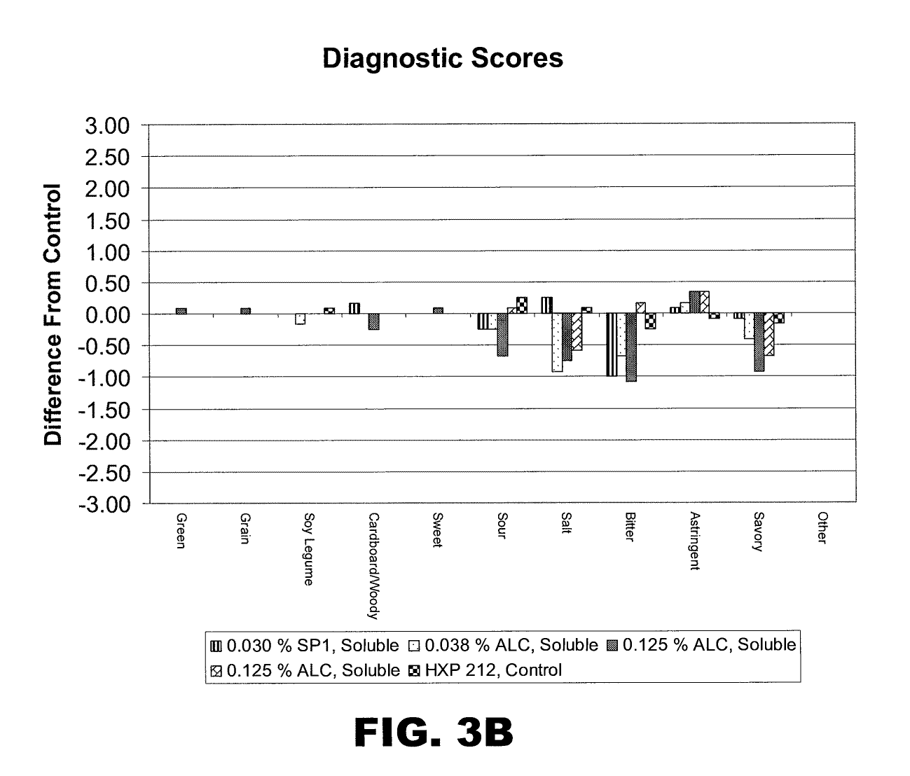

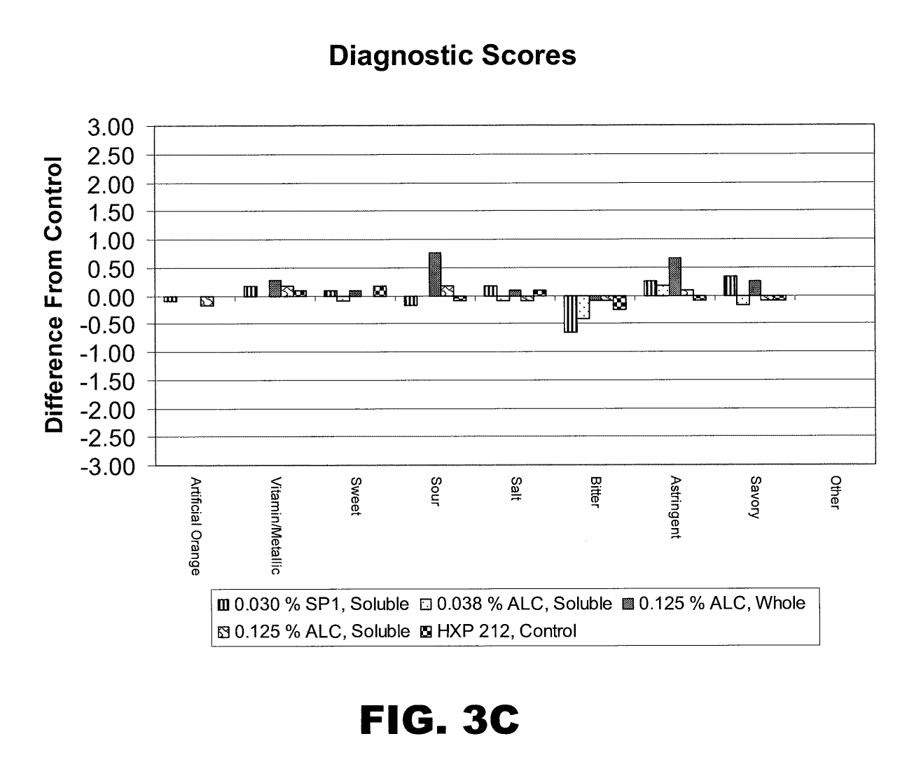

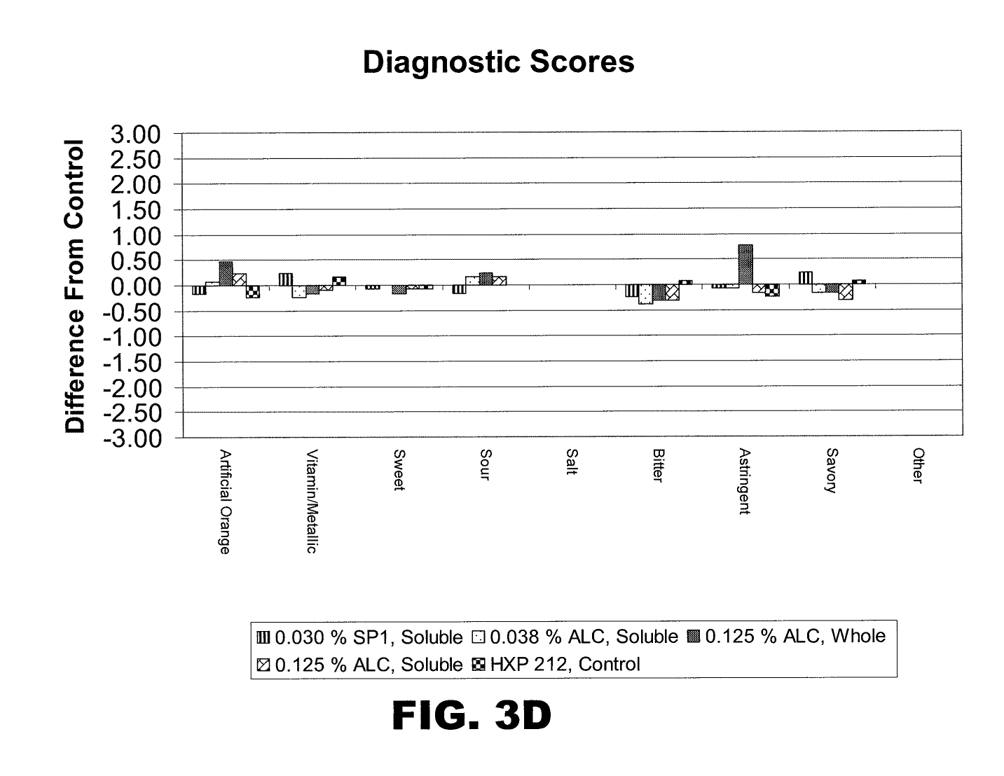

If a test sample was rated as different from the control sample (i.e., had an SQS score of 2, 3, or 4 as defined in Table 4), then the test sample was further evaluated to provide diagnostic information on how the test sample differed from the control sample. Thus, if the test sample had slightly more, moderately more, or extremely more of an attribute (as defined in Table 8 and shown in FIGS. 3A and 3B) than the control sample, then scores of +1, +2, +3, respectively, were assigned. Likewise, if the test sample had slightly less, moderately less, or extremely less of the attribute (as defined in Table 8 and shown in FIGS. 3A and 3B), than the control sample, then scores of −1, −2, −3, respectively, were assigned. This analysis provided an assessment of the directional quantitative differences between the test sample and the control sample.

TABLE 8
SQS Lexicon

| Attribute | Definition | References |
|---|---|---|
| Green | The general category of aromatics | Fresh cut grass, |
| | associated with green vegetation including | green beans, |
| | stems, grass, leaves and green herbs. | tomato vines |
| Grain | The aromatics associated with the total | All-purpose wheat |
| | grain impact, which may include all types of | flour in a water |
| | grain and different stages of heating. May | paste, cream of |
| | include wheat, whole wheat, oat, rice, | wheat, whole |
| | graham flour, etc. | wheat pasta |
| Soy/Legume | The aromatics associated with | Unsweetened |
| | legumes/soybeans; may include all types | SILK ™ soymilk, |
| | and different stages of heating. | canned |
| | | soybeans, tofu |
| Cardboard/Woody | The aromatics associated with dried wood | Toothpicks, water |
| | and the aromatics associated with slightly | from cardboard |
| | oxidized fats and oils, reminiscent of a | soaked for 1 hour |
| | cardboard box. | |
| Sweet | The taste on the tongue stimulated by | Sucrose |
| | sucrose and other sugars, such as fructose, | solutions: 2%, |
| | glucose, etc., and by other sweet | 5%, 10% |
| | substances, such as saccharin, Aspartame, | |
| | and Acesulfame-K. | |
| Sour | The taste on the tongue stimulated by acid, | Citric acid |
| | such as citric, malic, phosphoric, etc. | solutions: 0.05%, |
| | | 0.08%, 0.15% |
| Salt | The taste on the tongue associated with | Sodium chloride |
| | sodium salts. | solutions: 0.2%, |
| | | 0.35%, 0.5% |
| Bitter | The taste on the tongue associated with | Caffeine |
| | caffeine and other bitter substances, such | solutions: 0.05%, |
| | as quinine and hop bitters. | 0.08%, 0.15% |
| Astringent | The taste on the tongue associated with | MSG solution: |
| | monosodium glutamate. | 6.0% |

The directional differences of nine flavor attributes are presented in FIGS. 3A and 3B for hydrolysates with similar % DH levels. At all % DH levels, the TL1 hydrolysates had larger decreases in grain and soy/legume attributes and smaller increases in astringency and bitterness than did the ALC hydrolysates. The highest % DH ALC hydrolysates had particularly large increases in bitterness relative to the control.

First, the hydrolysates were presented to the assessors as 2.5% slurries in water at neutral pH, wherein the control was a SP1 hydrolysate. ALC and SP1 hydrolysates with about 12% DH and a commercially available soy hydrolysate (i.e., HXP 212) were compared with the SP1 hydrolysate control sample (see FIG. 3A). It was found that the ALC sample was slightly more bitter and salty than the control; the SP1 sample showed no difference, as expected; and the HXP 212 sample was slightly less salty, moderately more bitter, and slightly more savory than the control.

Next, the commercially available soy hydrolysate (HXP 212) was used as the control sample. Whole or soluble fractions of ALC and SP1 hydrolysates and the commercially available soy hydrolysate (HXP 212), as an internal control, were also compared to the HXP 212 control sample (see FIG. 3B). It was found that all of the ALC samples were slightly less salty than the control; the SP1 sample, the 0.038% ALC soluble fraction, and the 0.125% ALC whole fraction were all slightly less bitter than the control; and both 0.125% ALC samples were slightly less savory than the control.

Next, the hydrolysates were presented to the assessors in an orange sports drink at 1.6% protein and at pH 3.0 or pH 3.8, respectively (see FIGS. 3C and 3D). In both studies, the commercial soy hydrolysate (HXP 212) was used as the control. As shown in FIG. 3C, the SP1 soluble fraction was slightly less bitter than the control, and the 0.125% ALC whole fraction was slightly more sour and astringent than the control. The remaining samples in FIG. 3C showed no significant differences. In the study presented in FIG. 3D, the 0.125% ALC whole fraction was slightly more astringent than the control. The remaining samples in FIG. 3D showed no significant differences.

The results for FIGS. 3C and 3D are interpreted as per the attributes in Table 9

TABLE 9

| | | |
|---|---|---|
| Attribute | Definition | Reference |

AROMATICS

| | | |
|---|---|---|
| Overall Flavor | The overall intensity of the product | |
| Impact | aromas, an amalgamation of all | |
| | perceived aromatics, basic tastes and | |
| | chemical feeling factors. | |
| Artificial Orange | Aromatic associated with artificial | Baby Aspirin, 50/50 Ice |
| | orange | Cream Bars, Orange Fruit |
| | | Jelly Candy |
| Metallic | The aromatic associated with B- | B-complex vitamin, Vitamin/ |
| | vitamins and the aromatic associated | Mineral premix, Iron tablet, |
| | with metals, tin or iron. | canned tomato juice, |
| | | pennies |

BASIC TASTES

| | | |
|---|---|---|
| Sweet | The taste on the tongue stimulated by | Sucrose solutions: |
| | sucrose and other sugars, such as | 2%, 5%, 10% |
| | fructose, glucose, etc., and by other | |
| | sweet substances, such as saccharin, | |
| | Aspartame, and Acesulfame-K. | |
| Sour | The taste on the tongue stimulated by | Citric acid solutions: |
| | acid, such as citric, malic, phosphoric, | 0.05%, 0.08%, 0.15% |
| | etc. | |
| Salt | The taste on the tongue associated | Sodium chloride solutions: |
| | with sodium salts. | 0.2%, 0.35%, 0.5% |
| Bitter | The taste on the tongue associated | Caffeine solutions: |
| | with caffeine and other bitter | 0.05%, 0.08%, 0.15% |
| | substances, such as quinine and hop | |
| | bitters. | |

CHEMICAL FEELING FACTOR

| | | |
|---|---|---|
| Astringent | The shrinking or puckering of the | Alum solutions: |
| | tongue surface caused by substances | 0.005%, 0.007%, 0.01% |
| | such as tannins or alum. | |
| Savory/Umami | The taste on the tongue associated | MSG solution: |
| | with monosodium glutamate | 6.0% |

### Example 5

### Identification of Peptides in SP1 and ALCALASE® Hydrolysates

To further characterize the soy hydrolysates prepared with either SP1 or ALCALASE® (ALC), peptide fragments in the hydrolysates were identified by liquid chromatography mass spectrometry (LC-MS).

Samples were prepared by mixing an aliquot containing 3 mg of hydrolysate and 0.1% formic acid (300 μL) in a microcentrifuge tube and vortexing the mixture for 1-2 minutes. The entire mixture was then transferred to a pre-cleaned C18 tip (Glygen Corp., Columbia, Md.) for peptide isolation. The C18 tip was cleaned by eluting with 0.1% formic acid in 60% acetonitrile (300 μL) and equilibrated with 0.1% formic acid (600 μL). Materials eluted with 0.1% formic acid fraction were discarded, and the peptides were eluted with 0.1% formic acid in 60% acetonitrile (600 μL). Total volume of peptide solution was reduced to 200 μL by evaporating the solvent mixture in on Genevac EZ-2 evaporator at 30° C. for 10 minutes. An aliquot (25 μL) of the supernatant was injected into C18 analytical HPLC column (15 cm×2.1 mm id, 5 μm; Discovery Bio Wide Pore, Supelco, Sigma-Aldrich, St. Louis, Mo.) on a HP-1100 (Hewlett Packard; Palo Alto, Calif.) HPLC instrument. The elution profile is presented in Table 8. Solvent A was 0.1% formic acid; solvent B was 0.1% formic acid in acetonitrile, the flow rate was 0.19 mL/min, and the column thermostat temperature was 25° C.

TABLE 10
HPLC Solvent Elution Profile.

| Time | Solvent A | Solvent B |
|---|---|---|
| (min) | (%) | (%) |
| 0 | 95 | 5 |
| 35 | 55 | 45 |
| 37 | 55 | 45 |
| 39 | 10 | 90 |
| 42 | 10 | 90 |
| 44 | 95 | 5 |
| 45 | 95 | 5 |

An aliquot (10 μL) of the LC eluate was delivered to the ESI-MS source using a splitter system for MS analysis. A Thermo Finnigan LCQ-Deca ion trap mass spectrometer was used to analyze the peptides with data dependent MS/MS with dynamic exclusion scan events. ESI-MS was conducted at positive ion mode with capillary temperature 225° C., electrospray needle was set at a voltage 5.0 kV, and scan range from m/z 400-2000. The raw MS/MS data was deconvoluted by Sequest search engine (BIO WORKS™ software, Thermo Fisher Scientific, Pittsburgh, Pa.) with no enzyme search parameter. Peptides were identified by searching a standard database such as NCBI.

Table 11 presents the peptides identified in SP1 hydrolysates and Table 12 presents the peptides identified in ALC hydrolysates. A total of 37 distinct peptides were identified in the SP1 hydrolysates, 33 of which were unique to SP1 hydrolysates. A total of 11 peptides were identified in the ALC hydrolysates, 7 of which were unique to ALC hydrolysates.

TABLE 11
Peptides Identified in SP1 Hydrolysates.

| | | | SEQ |
|---|---|---|---|
| | | | ID |
| Protein | Peptide | MH+ | NO: |
| Alpha subunit of beta- | | | |
| conglycinin | | | |
| | SEDKPF | 722.25 | 2 |
| | FVDAQPK | 804.40 | 3 |
| | SAQAVEKL | 845.32 | 4 |
| | ISSEDKPF | 923.05 | 5 |
| | SRDPIYSN | 952.11 | 6 |
| | NQRSPQLQ | 970.99 | 7 |
| | AENNQRNF | 992.93 | 8 |
| | SRDPIYSNK | 1081.38 | 9 |
| | VNNDDRDSY | 1098.08 | 10 |
| | EITPEKNPQLR | 1325.33 | 11 |
| | FEITPEKNPQLR | 1471.85 | 12 |
| | SREEGQQQGEQRL | 1545.25 | 13 |
| Beta subunit of beta- | | | |
| conglycinin | | | |
| | FVDAQPQ | 804.40 | 14 |
| | SAQDVERLL | 1031.34 | 15 |
| | PGSAQDVERLL | 1185.14 | 16 |
| | AFPGSAQDVERLL | 1403.96 | 17 |
| Glycinin subunit G1 | | | |
| Proglycinin A1aB1b) | | | |
| | AEFGSL | 623.34 | 18 |
| | VSIIDTN | 761.29 | 19 |
| | PEEVIQH | 851.38 | 20 |
| | IQQGKGIF | 891.39 | 21 |
| | PEEVIQHTF | 1099.40 | 22 |
| | DGELQEGRVL | 1115.91 | 23 |
| | NALKPDNRIE | 1170.27 | 24 |
| | ALPEEVIQHTF | 1284.59 | 25 |
| | SLENQLDQMPR | 1330.69 | 26 |
| | NALPEEVIQHTF | 1398.87 | 27 |
| Glycinin Gy4A5A4B3 | | | |
| | DTSNF | 583.09 | 28 |
| | LDTSNF | 696.19 | 29 |
| | ADFYNPK | 854.31 | 30 |
| | HENIARPS | 923.85 | 31 |
| | NSQHPELK | 952.91 | 32 |
| | LHENIARPS | 1036.39 | 33 |
| | NNQLDQTPR | 1085.78 | 34 |
| | NTNEDIAEKL | 1146.35 | 35 |
| Glycinin A3B4 subunit | | | |
| | NSQHPELQ | 952.34 | 36 |
| | NTNEDTAEKL | 1134.82 | 37 |
| | RSPDDERKQIV | 1343.38 | 38 |

TABLE 12
Peptides Identified in ALC Hydrolysates.

| | | | SEQ |
|---|---|---|---|
| | | | ID |
| Protein | Peptide | MH+ | NO: |
| Alpha subunit of beta- | | | |
| conglycinin | | | |
| | SEDKPF | 722.28 | 2 |
| | AENNQRNF | 993.53 | 8 |
| Glycinin subunit G1 | | | |
| (Proglycinin A1aB1b) | | | |
| | AEFGSL | 623.31 | 18 |
| | SIIDTN | 662.39 | 39 |
| | VSIIDTN | 761.45 | 19 |
| | SQSDNFE | 826.28 | 40 |
| Glycinin Gy4A5A4B3 | | | |
| | DFYNPK | 783.44 | 41 |
| | LDQTPRVF | 975.99 | 42 |
| | NALEPDHRVE | 1180.18 | 43 |
| Glycinin A3B4 subunits | | | |
| | LDQNPRVF | 988.86 | 44 |
| | GNPDIEHPETM | 1239.35 | 45 |

### Example 6

### Molecular Weight Distribution of Peptide Fragments

Isolated soy protein was hydrolyzed with either SP1 or ALCALASE® (ALC) essentially as described above. SP1 was used at 1500 mg/kg soy, and ALC was used at 5.0% CBS (Curd solid basis). The degree of hydrolysis of the soluble fraction was determined as described above in Example 3. The degree of hydrolysis of the SP1 hydrolysate was 15.9% and the degree of hydrolysis of the ALC hydrolysate was 21.1%.

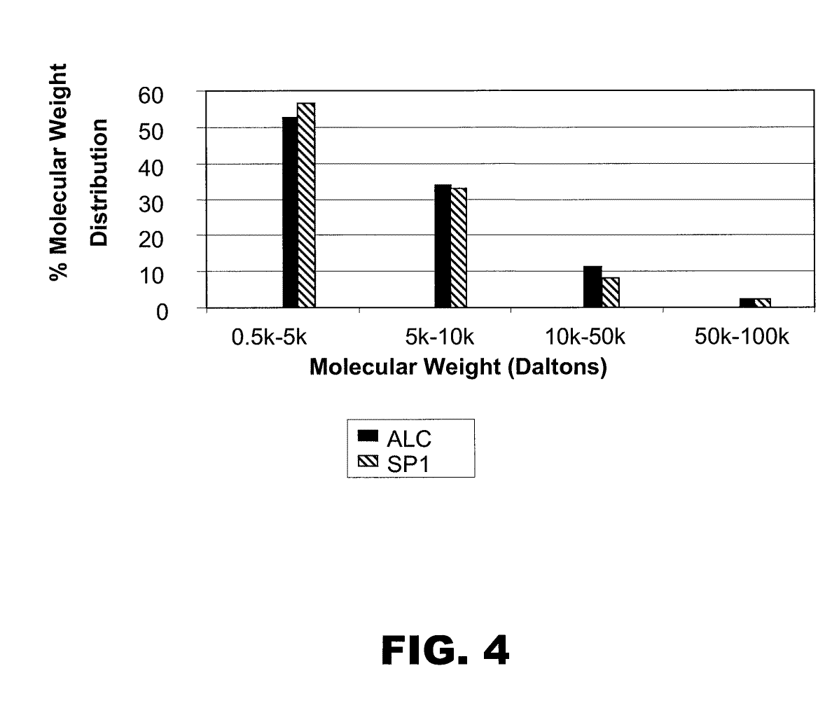

The molecular weight distribution of the peptide fragments in the SP1 and ALC hydrolysates was determined by size exclusion chromatography. The system was an Agilent 1100 HPLC series (Agilent Technologies, Santa Clara, Calif.) with a Zorbax GF-250 column and Zorbax guard column (Agilent Technologies) and a SPC GPEP-30 column (Eprogen Inc., Darien, Ill.). The mobile phases comprised phosphate buffered saline and 10% isopropanol. Protein standards ranging from 555 Daltons to 200,000 Daltons were also run. As shown in FIG. 4, the SP1 hydrolysate had more 500-5000 Dalton peptide fragments and fewer 10,000-50,000 Daltons fragments relative to the ALC hydrolysate.

### Example 7

### Energy Drink Prototypes

Prototypical orange sports beverages comprising SP1 or ALC soy hydrolysates were prepared and compared with an orange sports beverage comprising a commercial soy hydrolysate (i.e., HXP 212) with regards to flavor and functionality. Table 13 presents the compositions of the drinks. Each drink was split into two fractions, which were adjusted to pH 3.8 or pH 3.2. Each drink had about 4.0 grams of protein per 240 gram serving. The drinks were stored at 4° C.

TABLE 13
Sports Drink Formulations.

| | Control | SP1 | ALC |
|---|---|---|---|
| Ingredient | Drink | Drink | Drink |
| Water | 91.377% | 91.377% | 91.277% |
| HXP 212 | 1.980% | — | — |
| (84.3% protein) | | | |
| SP1 Hydrolysate | — | 1.980% | — |
| (84.2% protein) | | | |
| ALC Hydrolysate | — | — | 2.080% |
| (80.3% protein) | | | |
| Sugar | 3.800% | 3.800% | 3.800% |
| Fructose | 2.500% | 2.500% | 2.500% |
| Monopotassium | 0.040% | 0.040% | 0.040% |
| phosphate | | | |
| Orange dye | 0.300% | 0.300% | 0.300% |
| Yellow6 dye | 0.002% | 0.002% | 0.002% |
| Yellow5 dye | 0.0015% | 0.0015% | 0.0015% |
| % solids | 8.624% | 8.624% | 8.724% |

The viscosity of each drink was measured by using a viscometer (with spindle S61, speed at 60, and 4° C.) and the reading was taken at 1 minute. The turbidity was measured using a Turbiscan fixed position scan at 25 mm, with an average of 60 scans over 1 minute. Table 14 presents the viscosity (cP) and the turbidity (% Tm) of each sample. All of the drinks had acceptable viscosity measurements, but the drinks containing the ALC hydrolysate had low turbidity values.

TABLE 14
Viscosity and Turbidity Analysis.

| | |
|---|---|
| Viscosity | Turbidity |
| (cP) | (% Tm) |

pH 3.8 Set

| | | |
|---|---|---|
| Control | 3.7 | 2.08 |
| SP1 | 4.6 | 3.23 |
| ALC | 3.6 | 0.40 |

pH 3.2 Set

| | | |
|---|---|---|
| Control | 4.1 | 2.32 |
| SP1 | 4.2 | 6.70 |
| ALC | 3.3 | 0.65 |

The sediment of the different drinks was determined by placing the samples in 100 ml cylinders. The sediment was measured after one day or after two weeks (at 4° C.). Table 15 presents the percent of sediment of each. The drinks prepared with the ALC hydrolysate had much more sediment from the onset.

TABLE 15
Sediment Analysis.

| | |
|---|---|
| 24 hours | 2 weeks |
| Sediment (%) | Sediment (%) |

pH 3.8 Set

| | | |
|---|---|---|
| Control | 0.5 | 1.0 |
| SP1 | 0 | 0.5 |
| ALC | 2.0 | 2.5 |

pH 3.2 Set

| | | |
|---|---|---|
| Control | 0.5 | 1.0 |
| SP1 | 0 | 0.75 |
| ALC | 2.5 | 2.5 |

The flavor of the drinks was evaluated by a panel of five tasters. The tasters force ranked the drinks, which had been refrigerated for two weeks, from the most liked (score=1) to the least liked (scores=6). The sum of the scores for each drink is presented in Table 16. The drinks containing the SP1 hydrolysate had the most favorable liking scores.

TABLE 16
Flavor Analysis.

Total Score

pH 3.8 Set

| | |
|---|---|
| Control | 21.5 |
| SP1 | 19.5 |
| ALC | 27.5 |

pH 3.2 Set

| | |
|---|---|
| Control | 20.5 |
| SP1 | 15.0 |
| ALC | 16.0 |

In summary, this preliminary analysis of prototype acid drinks revealed that the SP1 hydrolysates performed very well.

What is claimed is:

1. A protein hydrolysate composition comprising a mixture of oligopeptides having an average size of less than about 10,000 Daltons, wherein the composition has a degree of hydrolysis of at least about 2.5% and a solid solubility index of at least about 60% at a pH of less than about 7.0.

2. The protein hydrolysate composition of claim 1, wherein the pH is less than about 5.0.

3. The protein hydrolysate composition of claim 1, wherein the solid solubility index is from about 70% to about 90%.

4. The protein hydrolysate composition of claim 1, wherein the degree of hydrolysis is about 10% to about 35%.

5. The protein hydrolysate composition of claim 1, wherein the protein hydrolysate composition is derived from a protein selected from the group consisting of soy, vegetable, animal, egg, and combinations thereof.

6. The protein hydrolysate composition of claim 1, wherein the protein hydrolysate composition is derived from soy in combination with at least one protein selected from the group consisting of vegetable, animal, dairy, and egg.

7. The protein hydrolysate composition of claim 1, wherein the protein hydrolysate composition is derived from soy.

8. The protein hydrolysate composition of claim 7, wherein the solid solubility index is about 70% to about 90% and the degree of hydrolysis is about 10% to about 35%.

9. The protein hydrolysate composition of claim 8, wherein the pH is less than about pH 5.0.

10. The protein hydrolysate composition of claim 6, wherein the composition comprises polypeptide fragments selected from the group consisting of at least 2 polypeptide fragments from SEQ ID NO: 2-38 or at least 1 polypeptide fragment for SEQ ID NO: 2-38 and at least 1 polypeptide fragment from SEQ ID NO: 39-45.

11. The protein hydrolysate composition of claim 7, wherein the composition comprises polypeptide fragments selected from the group consisting of at least 2 polypeptide fragments from SEQ ID NO: 2-38 or at least 1 polypeptide fragment for SEQ ID NO: 2-38 and at least 1 polypeptide fragment from SEQ ID NO: 39-45.

12. A process for preparing a protein hydrolysate composition, the process comprising:
a. contacting a protein material with at least one endopeptidase that cleaves peptide bonds of the protein material to form a mixture of oligopeptides having an average size of less than about 10,000 Daltons, the mixture of oligopeptides comprising the protein hydrolysate composition; and
b. lowering the pH of the protein hydrolysate composition to less than about pH 7.0, wherein the protein hydrolysate composition has a solid solubility index of at least about 60% and a degree of hydrolysis of at least about 2.5%.

13. The process of claim 12, wherein the endopeptidase is a food grade protease of microbial origin.

14. The process of claim 13, wherein the endopeptidase is selected from the group consisting of serine protease (SP1) from Nocardiopsis prasina, subtilisin protease from Bacillus licheniformis, and combinations thereof.

15. The process of claim 14, wherein the endopeptidase is SP1.

16. The process of claim 12, wherein the endopeptidase comprises an amino acid sequence that is at least 80% identical to SEQ ID NO:1.

17. The process of claim 16, wherein the endopeptidase comprises an amino acid sequence that is at least 90% identical to SEQ ID NO:1.

18. The process of claim 17, wherein the endopeptidase comprises an amino acid sequence that is at least 95% identical to SEQ ID NO:1.

19. The process of claim 18, wherein the endopeptidase comprises an amino acid sequence that is at least 99% identical to SEQ ID NO:1.

20. The process of claim 12, wherein the endopeptidase has optimal proteolytic activity at about pH 7.0 to about pH 11.0 and at a temperature of about 50° C. to about 80° C.

21. The process of claim 20, wherein the endopeptidase has optimal proteolytic activity at about pH 8.0 to about pH 9.0 and at a temperature of about 60° C. to about 70° C.

22. The process of claim 12, wherein about 20 mg to about 5000 mg of the endopeptidase is combined with each kilogram of protein material.

23. The process of claim 12, wherein the pH of the protein hydrolysate composition is lowered by the addition of an acid selected from the group consisting of citric acid, formic acid, fumaric acid, lactic acid, hydrochloric acid, malic acid, phosphoric acid, and combinations thereof.

24. The process of claim 12, wherein the pH of the protein hydrolysate composition is less than about pH 5.0.

25. The process of claim 12, wherein the protein material is selected from the group consisting of soy, barley, canola, lupin, maize, oat, pea, potato, rice, wheat, animal, egg, and combinations thereof.

26. The process of claim 12, wherein the protein material is soy in combination with at least one protein selected from the group consisting of barley, canola, lupin, maize, oat, pea, potato, rice, wheat, animal, dairy, and egg.

27. The process of claim 12, wherein the protein material is soy and the endopeptidase is SP1.

28. The process of claim 27, wherein the soy protein material is selected from the group consisting of soy extract, soymilk, soymilk powder, soy curd, defatted soy flour, partially defatted soy flour, full fat soy flour, isolated soy protein, soy protein concentrate, and combinations thereof.

29. The process of claim 28, wherein the solid solubility index of the soy protein hydrolysate composition is about 70% to about 90%, and the degree of hydrolysis of the soy protein hydrolysate composition is about 10% to about 35%.

30. The process of claim 29, wherein the pH of the soy protein hydrolysate composition is less than about pH 5.0.

31. The process of claim 26, wherein the protein hydrolysate composition comprises polypeptide fragments selected from the group consisting of at least 2 polypeptide fragments from SEQ ID NO: 2-38 or at least 1 polypeptide fragment for SEQ ID NO: 2-38 and at least 1 polypeptide fragment from SEQ ID NO: 39-45.

32. The process of claim 27, wherein the protein hydrolysate composition comprises polypeptide fragments selected from the group consisting of at least 2 polypeptide fragments from SEQ ID NO: 2-38 or at least 1 polypeptide fragment for SEQ ID NO: 2-38 and at least 1 polypeptide fragment from SEQ ID NO: 39-45.

33. A food product, the food product comprising:
a. an edible material; and
b. a protein hydrolysate composition, the composition comprising a mixture of oligopeptides having an average size of less than about 10,000 Daltons, wherein the composition has a degree of hydrolysis of at least about 2.5% and a solid solubility index of at least about 60% at a pH of less than about 7.0.

34. The food product of claim 33, wherein the protein hydrolysate composition is derived from a protein selected from the group consisting of soy, barley, canola, lupin, maize, oat, pea, potato, rice, wheat, animal, egg, and combinations thereof.

35. The food product of claim 33, wherein the protein hydrolysate composition is derived from soy in combination with at least one protein selected from the group consisting of barley, canola, lupin, maize, oat, pea, potato, rice, wheat, animal, dairy, and egg.

36. The food product of claim 33, wherein the protein hydrolysate composition is derived from soy, and the degree of hydrolysis is from about 10% to about 35%.

37. The food product of claim 33, wherein the food product is a beverage.

38. The food product of claim 37, wherein the beverage is a substantially clear beverage selected from the group consisting of a juice beverage, a fruit flavored beverage, a carbonated beverage, a sports drink, a nutritional supplement beverage, a weight management beverage, and an alcohol-based fruit beverage.

39. The food product of claim 37, wherein the beverage is a ready-to-drink beverage.

40. The food product of claim 37, wherein the beverage has a pH of less than about 5.0.

41. The food product of claim 37, wherein the beverage has a pH of less than about pH 4.0.

42. The food product of claim 37, wherein the beverage is a substantially cloudy beverage selected from the group consisting of a meal replacement drink, a protein shake, a coffee-based beverage, a nutritional supplement beverage, and a weight management beverage.

43. The food product of claim 37, wherein the edible material is selected from the group consisting of fruit juice, sugar, milk, non-fat dry milk powder, caseinate, soy protein concentrate, soy protein isolate, whey protein concentrate, whey protein isolate, chocolate, cocoa powder, coffee, and combinations thereof.

44. The food product of claim 37, wherein the food product further comprises an ingredient selected from the group consisting of a sweetening agent, an emulsifying agent, a thickening agent, a stabilizer, a lipid material, a preservative, an antioxidant, a flavoring agent, a coloring agent, a vitamin, a mineral, and combinations thereof.
